(19) United States
(12) Patent Application Publication
(10) Pub. No.: US 2026/0365957 A1
(43) Pub. Date: Nov. 19, 2026

(54) CELL CULTURE DEVICE INCLUDING AT LEAST ONE NOTCH FORMED IN A SIDEWALL FOR EXCHANGE OF FLUID MEDIA

(71) Applicant: CORNING INCORPORATED, CORNING, NY (US)

(72) Inventors: Audrey Blanche Bergeron, Haverhill, MA (US); Thomas Albert Cloutier, Arundel, ME (US); Hannah Jane Gitschier, Kennebunk, ME (US); Amy Claire Kauffman, Hightstown, NJ (US); William Joseph Lacey, North Andover, MA (US); Ana Maria del Pilar Pardo, Portsmouth, NH (US)

(21) Appl. No.: 19/133,611

(22) PCT Filed: Nov. 8, 2023
(86) PCT No.: PCT/US2023/036994

§ 371 Date: May 28, 2025

Related U.S. Application Data
(60) Provisional application No. 63/428,142, filed on Nov. 28, 2022.

(51) Int. Cl. C12M 1/00; C12M 1/32

(52) U.S. Cl. CPC C12M 29/00; C12M 23/12

(57) ABSTRACT

Embodiments of the disclosure relate to a device for three-dimensional cell growth. The device includes a substrate having an ultra-low attachment surface and a plurality of cavities for growing cell cultures. The device further includes at least one sidewall surrounding the substrate. At least one notch is formed in the at least one sidewall, and the at least one notch configured to accommodate a serological pipette for exchange of fluid media.

### CROSS-REFERENCE TO RELATED APPLICATIONS

This application claims the benefit of priority under 35 U.S.C. § 119 of U.S. Provisional Application Ser. No. 63/428,142 filed on Nov. 28, 2022, the content of which is relied upon and incorporated herein by reference in its entirety.

### BACKGROUND

The disclosure relates generally to a cell culture device and more particularly to a cell culture device that contains a microcavity substrate bottom including a feature that limits disruption of cell cultures during exchange of fluid.

Three-dimensional (3D) cell culture models have gained attention for better mimicking the complex microenvironment of cells and tissues that occur in vivo compared to two-dimensional (2D) monolayer cell culture methods. As biological research, cancer research, and the development of regenerative therapies progresses, there has been increased demand for generating greater quantities of 3D cell cultures, including cell aggregates, tumor spheroids, embryoid bodies, and organoids. Cell culture devices are available that allow for generating multiple 3D cell cultures in the same assay plate. Indeed, certain microplates allow for the formation of thousands of reproducibly sized and shaped 3D cell cultures in a traditional assay plate footprint by incorporating a microcavity substrate with an ultra-low attachment surface (ULA) in an open frame. In order to sustain 3D cell culture over the days and weeks necessary for the formation of multicellular 3D models and organoids, it is necessary to perform multiple rounds of fluid media exchanges while retaining the cells within the microcavity structures with minimal disturbance. For some cell culture devices, media exchanges can be performed without disturbing the 3D cultures as only a small volume (μ-mL range) of liquid is needed to fill the cavity and support the cultures. However, for certain high-density cell culture devices, moving larger volumes of liquid (e.g., 25 mL) is necessary to support the bulk 3D cell culture quantities present across the device. The movement of such comparatively high volumes of fluid may undesirably disturb the 3D cell cultures.

### SUMMARY

According to aspect (1), a device for three-dimensional cell growth is provided. The device comprising: a substrate comprising an ultra-low attachment surface and a plurality of cavities for growing cell cultures; and at least one sidewall surrounding the substrate; wherein at least one notch is formed in the at least one sidewall, the at least one notch configured to accommodate a serological pipette for exchange of fluid media.

According to aspect (2), the device of aspect (1) is provided, wherein the at least one sidewall comprises a first short sidewall, a second short sidewall, a first long sidewall, and a second long sidewall, wherein the first short sidewall and the second short sidewall each comprise a first length, and wherein the first long sidewall and the second long sidewall each comprise a second length, the second length being greater than the first length.

According to aspect (3), the device of aspect (2) is provided, wherein the at least one notch comprises a first notch formed on the first short sidewall.

According to aspect (4), the device of aspect (3) is provided, wherein the first notch is located at a midpoint of the first short sidewall.

According to aspect (5), the device of aspect (3) or (4) is provided, wherein the at least one notch comprises a second notch formed at a corner between the first short sidewall and the first long sidewall.

According to aspect (6), the device of aspect (2) is provided, wherein the at least one notch comprises a first notch formed at a first corner between the first short sidewall and the first long sidewall.

According to aspect (7), the device of aspect (6) is provided, wherein the at least one notch comprises a second notch formed at a second corner between the first short sidewall and the second long sidewall.

According to aspect (8), the device of any of the preceding aspects is provided, wherein the at least one notch comprises substantially planar notch walls.

According to aspect (9), the device of any of aspects (1)-(7) is provided, wherein the at least one notch comprises a radiused notch wall.

According to aspect (10), the device of any of the preceding aspects is provided, wherein the at least one notch comprises a floor, wherein the floor is angled relative to a plane defined by the substrate.

According to aspect (11), the device of aspect (10) is provided, wherein the floor is at a different height than the plane defined by the substrate.

According to aspect (12), the device of any of the preceding aspects is provided, wherein the plurality of cavities is at least 5 cavities per cm² of the substrate.

According to aspect (13), the device of any of the preceding aspects is provided, wherein the at least one notch is configured to accommodate the tip of a 25 mL serological pipette oriented perpendicularly to the substrate within the at least one notch.

According to aspect (14), the device of any of the preceding aspects is provided, wherein the device comprises dimensions compliant with ANSI/SLAS 1-2004.

According to aspect (15), the device of aspect (1) is provided, wherein the at least one sidewall is a single circular sidewall.

According to aspect (16), a method is provided. The method comprising: inserting a pipette into a first notch formed in at least one sidewall, the at least one sidewall surrounding a substrate of a cell culture device, the substrate comprising an ultra-low attachment surface and a plurality of cavities for growing cell cultures; and adding a fluid into the first notch such that the fluid covers the substrate within the at least one sidewall.

According to aspect (17), the method of aspect (16) is provided, wherein the at least one sidewall comprises a first short sidewall, a second short sidewall, a first long sidewall, and a second long sidewall, wherein the first short sidewall and the second short sidewall each comprise a first length, and wherein the first long sidewall and the second long sidewall each comprise a second length, the second length being greater than the first length.

According to aspect (18), the method of aspect (17) is provided, wherein inserting further comprises inserting the pipette into the first notch, the first notch being formed on the first short sidewall.

According to aspect (19), the method of aspect (18) is provided, further comprising removing the fluid from over the substrate using a pipette inserted into the first notch.

According to aspect (20), the method of aspect (19) is provided, wherein prior to removing the method further comprises raising the second short sidewall, the second short sidewall being opposite to the first short sidewall, such that the fluid flows toward the first notch formed on the first short sidewall.

According to aspect (21), the method of any of aspects (18)-(20) is provided, wherein the first notch is located at a midpoint of the first short sidewall.

According to aspect (22), the method of aspect (17) is provided, wherein the cell culture device further comprises a second notch and wherein one of the first notch or the second notch is formed on the first short sidewall and the other of the first notch or the second notch is formed at a corner between the first short sidewall and the first long sidewall.

According to aspect (23), the method of aspect (22) is provided, further comprising removing the fluid from over the substrate using a pipette inserted into the second notch.

According to aspect (24), the method of aspect (22) is provided, the method further comprises raising the second short sidewall, the second short sidewall being opposite to the first short sidewall, such that the fluid flows toward at least one of the first notch or the second notch.

According to aspect (25), the method of aspect (17) is provided, wherein inserting further comprises inserting the pipette into the first notch, the first notch being formed at a first corner between the first short sidewall and the first long sidewall.

According to aspect (26), the method of aspect (25) is provided, wherein the cell culture device further comprises a second notch formed at a second corner between the first short sidewall and the second long sidewall and wherein the method further comprises removing the fluid from over the substrate using a pipette inserted into the second notch.

According to aspect (27), the method of any of aspects (16)-(26), wherein the at least one notch comprises substantially planar notch walls.

According to aspect (28), the method of any of aspects (16)-(26), wherein the at least one notch comprises a radiused notch wall.

According to aspect (29), the method of any of aspects (16)-(28), wherein the at least one notch comprises a floor, wherein the floor is angled relative to a plane defined by the substrate.

According to aspect (30), the method of aspect (29) is provided, wherein the floor is at a different height than the plane defined by the substrate.

According to aspect (31), the method of any of aspects (16)-(30), wherein the plurality of cavities is at least 5 cavities per cm² of the substrate.

According to aspect (32), the method of any of aspects (16)-(31), wherein the at least one notch is configured to accommodate a 25 mL serological pipette oriented perpendicularly to the substrate within the at least one notch.

According to aspect (33), the method of any of aspects (16)-(32), wherein the cell culture device comprises dimensions compliant with ANSI/SLAS 1-2004.

According to aspect (34), the method of aspect (16) is provided, wherein the at least one sidewall is a single circular sidewall.

Additional features and advantages will be set forth in the detailed description which follows, and in part will be readily apparent to those skilled in the art from the description or recognized by practicing the embodiments as described in the written description and claims hereof, as well as the appended drawings.

It is to be understood that both the foregoing general description and the following detailed description are merely exemplary, and are intended to provide an overview or framework to understand the nature and character of the claims.

### BRIEF DESCRIPTION OF THE DRAWINGS

The accompanying drawings are included to provide a further understanding and are incorporated in and constitute a part of this specification. The drawings illustrate one or more embodiment(s), and together with the description serve to explain principles and operation of the various embodiments. In the drawings:

FIG. 1A depicts an open-well cell culture device including two notches formed at corners of the sidewalls of the device, according to an exemplary embodiment;

FIG. 1B depicts a detail view of the cavities of the cell culture device of FIG. 1A, according to an exemplary embodiment;

FIG. 2 depicts a cell culture device containing a fluid media, according to an exemplary embodiment;

FIG. 3 depicts a cell culture device having a lid and a tray, according to an exemplary embodiment;

FIG. 4 depicts a cell culture device with one notch formed in a sidewall of the device, according to an exemplary embodiment;

FIG. 5A depicts a cell culture device with one notch formed in a sidewall of the device and another notch formed at a corner of the sidewalls of the device, according to an exemplary embodiment;

FIG. 5B depicts a detail view of a corner notch, such as the corner notch shown in FIG. 5A, according to an exemplary embodiment;

FIG. 6 depicts a notch having a radiused wall, according to an exemplary embodiment;

FIG. 7 depicts a notch having planar walls, according to an exemplary embodiment;

FIG. 8 depicts a cross-sectional view of a notch and the substrate showing an angle of the notch floor relative to the plane of the substrate, according to an exemplary embodiment;

FIG. 9 depicts an embodiment of a multi-well cell culture device having a notch provided for each well, according to an exemplary embodiment; and

FIG. 10 depicts a process flow diagram for a method of adding and removing fluid media from a cell culture device, according to an exemplary embodiment.

### DETAILED DESCRIPTION

Referring generally to the following description and appended figures, various embodiments of a cell culture device having at least one notch formed in a sidewall are provided. As will be described more fully below, the notch formed in the sidewall of the cell culture device accommodates a pipette for adding and removing fluid (such as media, etc.) from the cell culture device. Such devices may be used for growing scaffold-free three-dimensional cell cultures (such as cell aggregates, spheroids, organoids, etc.) in cavities of a substrate of the cell culture device. Advantageously, the notch allows for exchange of fluid while decreasing the disturbance to the growing cell cultures. That is, exchange of fluid for replenishing of nutrients and removal of waste is typically associated with some level of cell culture loss as a result of the flow of the fluid over the substrate. By providing a notch where the fluid can be added into and removed from the microplate, the loss of cell cultures during an exchange of fluid is reduced. These and other aspects and advantages of the disclosed cell culture device and method of using same will be described herein and in relation to the figures. Such exemplary embodiments are provided by way of illustration and not by way of limitation.

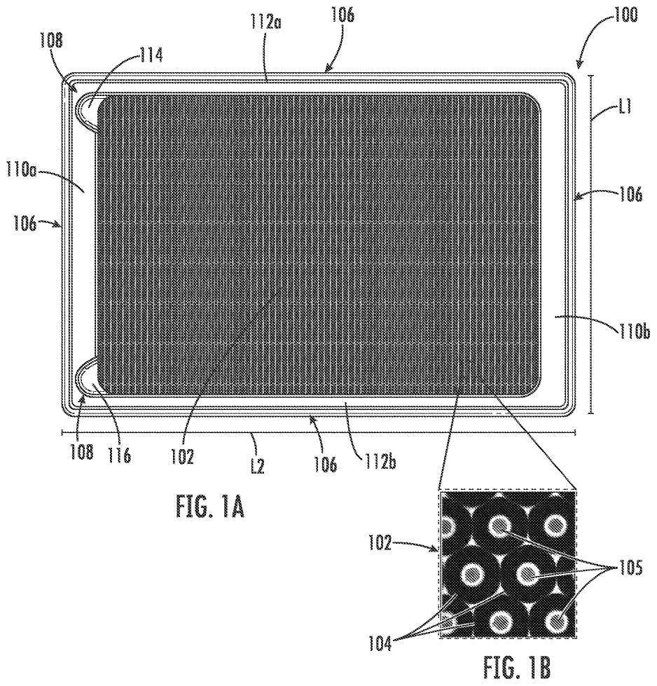

FIG. 1A depicts one embodiment of a cell culture device, in particular a plate 100, such as an open-well plate, configured for three-dimensional cell growth. The plate 100 may be a microplate. The open-well plate 100 comprises a substrate 102 having a plurality of cavities 104 (as shown in FIG. 1B) for growing cell cultures, such as cell aggregates, spheroids, and organoids, among others. For example, in one or more embodiments, the plurality of cavities 104 is at least 5 cavities per cm² of substrate. FIG. 1B provides a magnified view of the substrate 102 depicting the cavities 104 containing a cell culture, in particular a spheroid 105, in each cavity 104. In the particular embodiment shown in FIG. 1B, the cavities 104 have a diameter of around 800 um. In one or more embodiments, the substrate 102, in particular the cavities 104, are coated with an ultra-low attachment surface (ULA), which refers to a class of coatings known in the art to force cells into a suspended state.

Returning to FIG. 1A, the open-well plate 100 further includes a plurality of sidewalls 106 forming a perimeter that surrounds the substrate 102. At least one notch 108 is formed in the plurality of sidewalls 106. In one or more embodiments, the at least one notch 108 configured to accommodate a pipette, such as a serological pipette, for exchange of fluid. For example, in one or more embodiments, the at least one notch 108 is configured to accommodate a 25 mL serological pipette oriented perpendicularly to the substrate 102 within the at least one notch 108.

In one or more embodiments, the plurality of sidewalls 106 includes a first short sidewall 110a, a second short sidewall 110b, a first long sidewall 112a, and a second long sidewall 112b. The first short sidewall 110a and the second short sidewall 110b each have a first length L1, and the first long sidewall 112a and the second long sidewall 112b each have a second length L2. In one or more embodiments, the second length L2 is greater than the first length L1. In one or more embodiments, the plurality of sidewalls 106 define a rounded rectangular perimeter of the substrate 102. In one or more embodiments, the dimensions of the open-well plate 100 are compliant with ANSI/SLAS 1-2004. While a particular embodiment and shape of an open-well plate 100 are described and depicted herein, other shapes and sizes are also encompassed by the present disclosure.

In one or more embodiments, the at least one notch 108 includes a first notch 114 and a second notch 116. In one or more embodiments, the first notch 114 is formed at a corner between the first short sidewall 110a and the first long sidewall 112a. In one or more embodiments, the second notch 116 is formed at a corner between the first short sidewall 110a and the second long sidewall 112b. As mentioned, the notches 108 are provided to facilitate exchange of fluid for the substrate 102. For example, the fluid may contain nutrients to grow cell cultures, and thus, the fluid may be replenished as nutrients are depleted. Further, old fluid may be removed to remove waste from the cell cultures in the fluid.

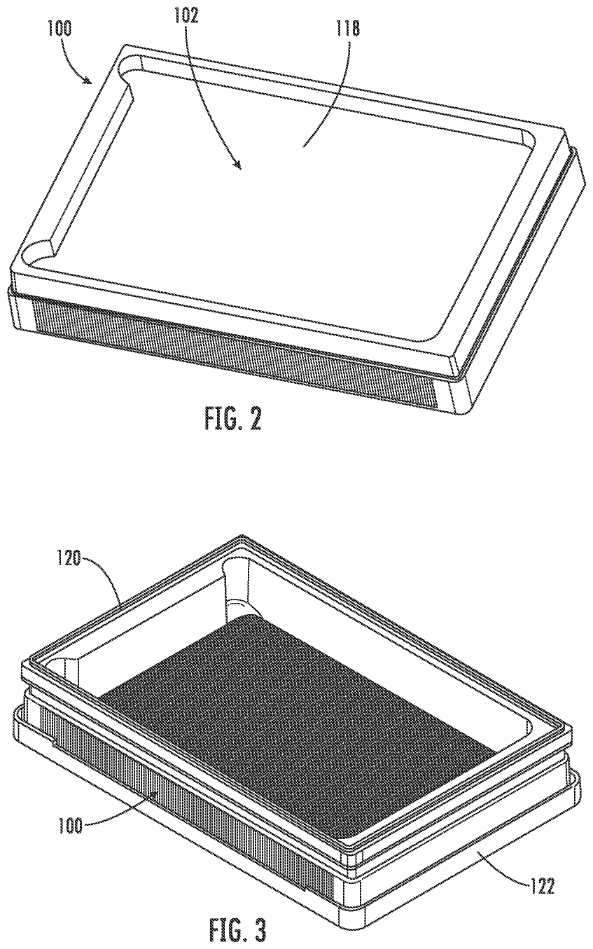

FIG. 2 depicts an example of an open-well plate 100 in which the substrate 102 is covered with a fluid 118. While the cell cultures are growing in the cavities 104 of the substrate 102, the open-well plate 100 may be configured for storage or handling. Thus, as shown in FIG. 3, the open-well plate 100 is provided with a lid 120 and a tray 122. In one or more embodiments, the lid 120 is provided to prevent contamination and spillage. In one or more embodiments, such as the embodiment shown in FIG. 3, the lid 120 is made of a transparent material such that the contents of the open-well plate 100 can be viewed through the lid 120. In one or more embodiments, the tray 122 provides a flat and stable surface for the open-well plate 100.

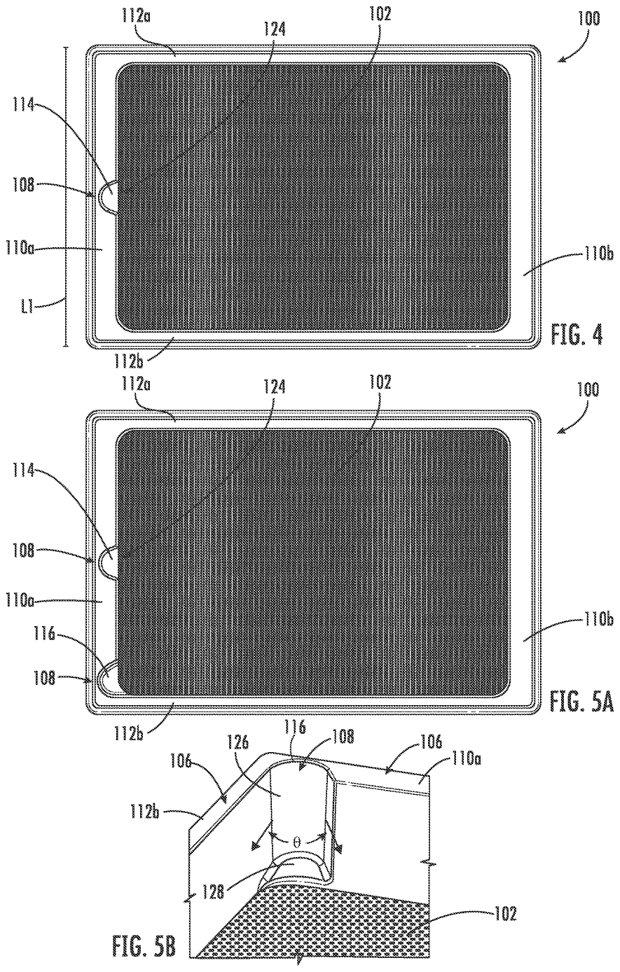

FIG. 4 depicts another embodiment of an open-well plate 100. In one or more embodiments, the at least one notch 108 of the open-well plate 100 is just a first notch 114 formed on the first short sidewall 110a. In one or more embodiments, including the embodiment depicted in FIG. 4, the first notch 114 is located at approximately the midpoint 124 of the first short sidewall 110a. In one or more embodiments, the midpoint 124 at which the first notch 114 is centered is a point between 40% and 60% of the first length L1 of the first short sidewall 110a (i.e., between 0.4L1 and 0.6L1).

FIG. 5A depicts another embodiment of an open-well plate 100 having two notches 108. As with the previous embodiment, the first notch 114 is provided at about the midpoint 124 of the first short sidewall 110a. The second notch 116 is provided at a corner between the first short sidewall 110a and the second long sidewall 112b.

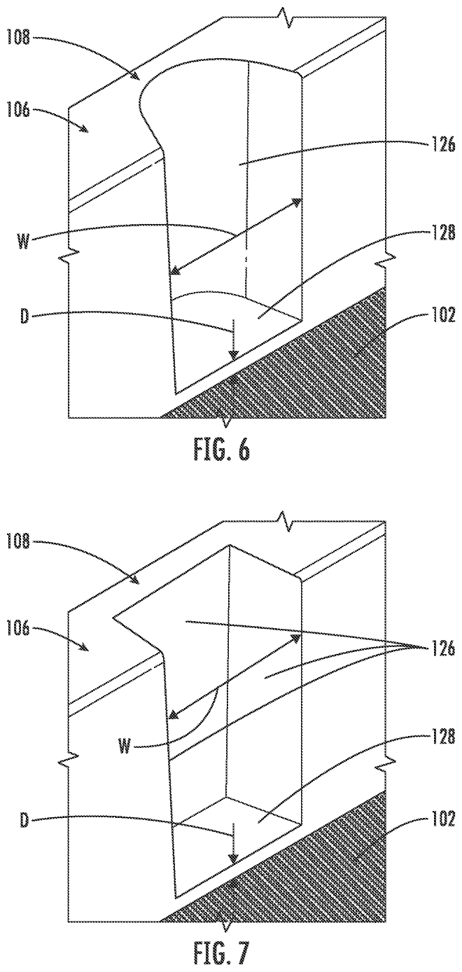

FIG. 6 depicts an embodiment of a notch 108 having a radiused notch wall 126. In one or more embodiments, the radiused notch wall 126 is curved at a radius of curvature of from 3.0 mm to 4.2 mm. Further, the notch 108 has a width W and depth (as defined by the radius of curvature) configured to accommodate a pipette, such as a 25 ml serological pipette. In one or more embodiments, the width W is from 5 mm to 15 mm, in particular from 8 mm to 11 mm. The notch 108 also has a notch floor 128. In one or more embodiments, the notch floor 128 is at a different height than the substrate 102. In one or more embodiments, the notch floor 128 is spaced a distance D above the substrate 102. In one or more embodiments, the distance D is from 0.5 mm to 1.5 mm. In one or more embodiments, the notch 108 with radiused notch walls 126 can be either or both of the first notch 114 and the second notch 116 of any of the embodiments of the open-well plates 100 described herein.

FIG. 7 depicts an embodiment of a notch 108 including a plurality of substantially planar notch walls 126. In one or more embodiments, the notch 108 has three planar notch walls 126 defining a rectangular notch 108. However, in one or more other embodiments, the notch 108 could have two planar notch walls 126 or more than three planar notch walls 126. In or more embodiments, the notch 108 has a width W and depth configured to accommodate a pipette, such as a 25 ml serological pipette. In one or more embodiments, the width W is from 5 mm to 15 mm, in particular from 8 mm to 11 mm. The notch 108 also has a notch floor 128. In one or more embodiments, the notch floor 128 is spaced a distance D above the substrate 102, such as a distance D of from 0.5 mm to 1.5 mm. In one or more embodiments, the notch 108 with substantially planar notch walls 126 can be either or both of the first notch 114 and the second notch 116 of any of the embodiments of the open-well plates 100 described herein.

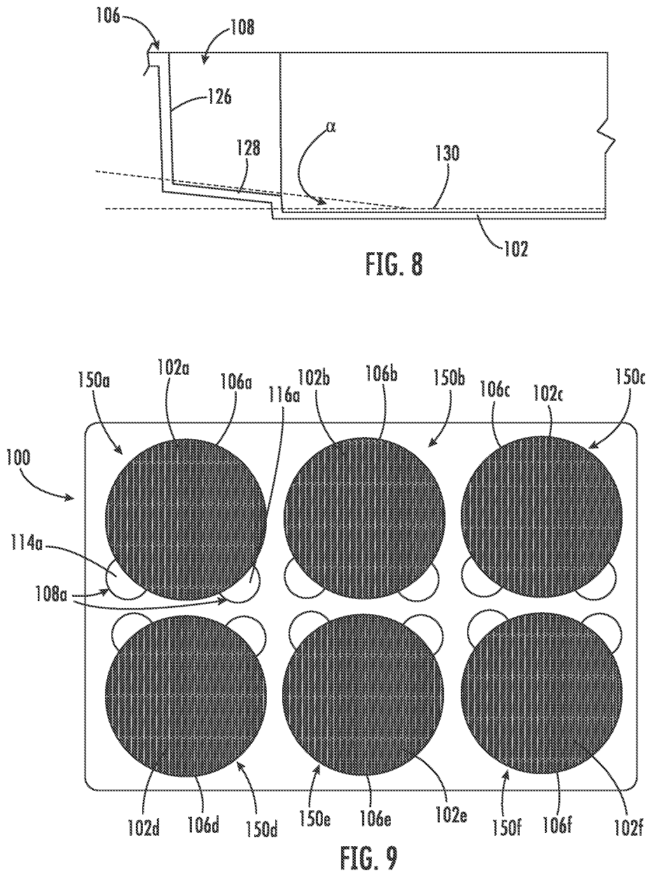

FIG. 5B depicts a particular embodiment of a corner notch 108 having a radiused notch wall 126. For the purpose of discussion, reference will be made to the second notch 116 of FIG. 5A, but the corner notch 108 could be located in any corner. As can be seen in FIG. 5B, the second notch 116 is formed at the intersection of first short sidewall 110a and the second long sidewall 112b. Further, in one or more embodiments, the second notch 116 opens at an angle θ of, e.g., 20° to 40°, in particular about 34°, relative to the second long sidewall 112b. Because of the angled opening of the second notch 116, the edge of the second notch 116 against the second long sidewall 112b is longer than the edge of the second notch 116 provided on the first short sidewall 110a. Further, as will be described more fully in relation to FIG. 8, the notch floor 128 of the second notch 116 of FIG. 5B is sloped toward the substrate 102. Because of the slope of the notch floor 128 and the longer extension of one edge of the second notch 116, fluid added at the second notch 112 will flow under the influence of surface tension toward the lowest point of the second notch 116, which is toward the edge formed with the second long sidewall 112b. In this way, the fluid added to the second notch 116 is directed away from the substrate and toward the second long sidewall 112b. Advantageously, this helps to decrease the disturbance that the addition of liquid has on the cells growing in the substrate 102.

FIG. 8 depicts a cross-sectional view of a notch 108 in relation to the substrate 102. As can be seen in FIG. 8, the substrate 102 defines a plane 130. In one or more embodiments, the notch floor 128 forms an angle α with the plane 130. In one or more embodiments, the angle a is up to 15°, in particular up to 11°. Advantageously, the angled notch floor 128 helps encourage the flow of fluid media from the notch 108 into the region over the substrate 102. In particular, when combined with the angled opening of a corner notch (as described in relation to FIG. 5B), the fluid indirectly flows into the region over the substrate 102 after first being drawn toward a sidewall 106.

While the foregoing embodiments have focused on an open-well plate 100 having sidewalls 106 defining a rectangular perimeter of a single substrate 102, other embodiments of the device may include multiple isolated compartments (i.e. microwells, multiple wells, alternative dish, or reservoir geometries) each with their own microcavity substrate bottom. According to an example embodiment shown in FIG. 9, the plate 100 contains multiple wells 150a-f, each with a corresponding substrate 102a-f. Each well 150a-f is defined by a single corresponding sidewall 106a-f (circular sidewall), and each sidewall 106a-f includes at least one notch for adding and removing fluid from the wells 150a-f. In one or more embodiments, each well 150a-f includes two notches, a first notch for adding fluid media and a second notch for removing fluid media (for ease of illustration only the notches 108a, including first notch 114a and second notch 116a, of the well 150a are labeled). The wells 150a-f can be used to grow different cell cultures in each well and/or to grow the same cell cultures in each well under different conditions, amongst other possibilities.

FIG. 9 depicts plate 100 having multiple substrates 102, but other embodiments may have a single substrate 102 with a single sidewall 106 (e.g., a circular dish), amongst other possibilities.

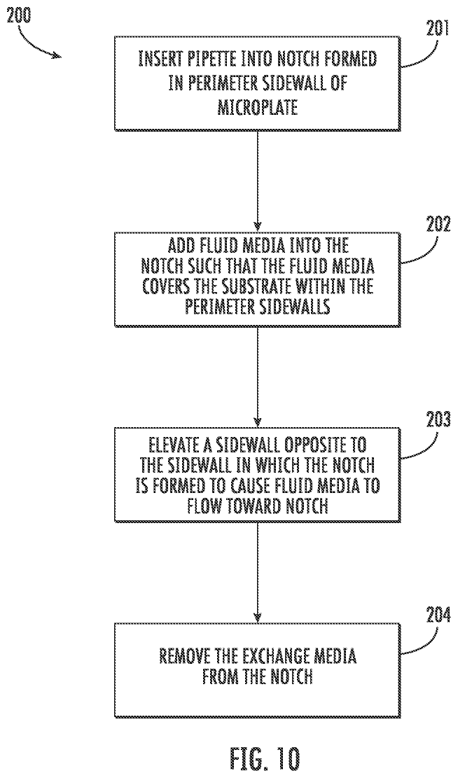

Embodiments of the disclosure also relate to a method of adding and removing fluid from a plate, such as a microplate. FIG. 10 provides a process flow diagram of the method 200, which will be described in relation to the embodiments of the plates 100 depicted in FIGS. 1A-5A (although the method 200 applies to any cell culture device having notches for adding or removing fluid). In one or more embodiments, the method 200 includes a first step 201 of inserting a pipette into a notch 108 formed in a perimeter sidewall 106 of the open-well plate 100.

In one or more embodiments, the method 200 further includes a second step 202 of adding a fluid into the notch 108 such that the fluid covers the substrate 102 within the plurality of sidewalls 106. In one or more embodiments, the pipette is arranged perpendicularly to the substrate 102 such that the fluid is directed substantially at the notch floor 128. However, in one or more other embodiments, the pipette is arranged transversely to the plane of the substate 102 such that the fluid is directed at least partially against the notch wall 126. Once the fluid covers the substrate 102, the open-well plate 100 may be stored in a controlled environment to permit growth of the cell cultures.

After a desired amount of time has passed and the fluid media needs to be exchanged, the plate 100 may be retrieved for removal of the fluid media. To facilitate removal of the fluid media, in one or more embodiments, the method 200 further includes an optional third step 203 of elevating a sidewall 106 of the plate 100 opposite to the notch 108 such that the fluid media flows toward the notch 108 (either a first notch 114 or a second notch 116) formed in the perimeter sidewalls 106 of the plate 100. In experimentation, the inventors have found that elevating one side of the plate 100 facilitates removal of the fluid media by causing it to drain to the opposite side of the device. However, the inventors also found that the notch 108 permitted removal of over 50% of the fluid media even when one side was not elevated for draining the fluid to the opposite side.

In one or more embodiments, the method 200 further includes a fourth step 204 of removing the fluid media from the notch 108 (either from a first notch 114 or from a second notch 116) formed in the perimeter sidewall 106 of the plate 100. The method 200 can be repeated until the cell cultures have grown to a desired level.

Advantageously, embodiments of the device provide a convenient way to exchange fluid media without substantial disturbance to the cell cultures. In this regard, the inventors envision that exchange of fluid media can be performed while retaining ≥90% cell yield from the device. Absent the notch 108 or notches 108 formed in the sidewall 106, fluid media addition or removal would occur directly over the cell culture substrate 102 or against one of the sidewalls 106, resulting in extensive cell disturbance and displacement. The inventors have observed that cell culture yields of less than 90%, often much less than 90%, are typical of exchanges using conventional cell culture devices. The at least one notch 108, however, allows for consistent placement of pipettes for fluid media exchange. Indeed, random pipette placement during fluid media exchange can result in inconsistent cultures and results. Further, having multiple notches 108 accommodates for both left-handed and right-handed operators while providing consistent pipette placement during fluid media exchange.

Various embodiments of the plate 100 were tested to determine the ease with which fluid media could be exchanged and how much the cell cultures were disturbed.

In a first experiment, open-well plates 100 were prepared (1) having two notches 108 with a first notch 114 at a first corner of a side and a second notch 116 at the second corner of the side, (2) having a single notch 108 located at a midpoint of a side, (3) having two notches 108 with a first notch 114 at a midpoint of a side and a second notch 116 located at a corner of the side, and (4) having a single notch 108 located at one corner.

With respect to the first open-well plate 100, 13 mL of fluid was added at the first notch 114, and during fluid removal, 11 mL of the fluid was able to be removed while the open-well plate 100 remained flat. It was observed that the two notches 108 were beneficial for lefthand and righthand operators, in particular to access the front or back of the open-well plate as desired.

With respect to the second open-well plate 100, 13 mL of fluid was added at the single side notch 108, and during removal, 7.5 mL of fluid was able to be removed while the open-well plate 100 remained flat.

With respect to the third open-well plate 100, 13 mL of fluid was added at the second notch 116 located in the corner, and during removal, only a few milliliters were able to be removed from either the first notch 114 or the second notch 116. When the side opposite to the notches was elevated about 3°, then 10.2 mL was able to be removed from the second notch 116.

With respect to the fourth open-well plate 100, 13 mL of fluid was added at the single corner notch 108, and during removal, only 5 mL of fluid was able to be removed while the plate 100 remained flat. It was observed that the radiused notch wall 126 located in the corner was able to comfortably accommodate the pipette tip.

In a second experiment, microplates were prepared having three notches 108 formed in the sidewall 106. In particular, the first short sidewall 110a included notches 108 at each corner and a notch 108 at the midpoint 124. The plate 100 was prepared by providing a ULA surface. Trapped air from microcavities was removed by centrifugation. Thereafter, cavities of the substrate close to the first short sidewall 110a were populated with pre-formed spheroids. The plates were imaged prior to addition or removal of fluid media. To evaluate spheroid disturbance, 10 mL of fluid medium was added and then removed. During removal, the second short sidewall 110b was elevated by about 3°. Three fluid exchange conditions were considered: (1) addition and removal of fluid from the same corner notch 108, (2) addition of fluid at the midpoint notch 108 and removal from a corner notch 108, and (3) addition of fluid at one corner notch 108 and removal of fluid from the other corner notch 108. After each fluid addition and each fluid removal, the plates 100 were imaged using a Nikon NEXIV vision measurement system (VMR).

Five different operators performed the fluid addition and removal steps outlined above, and it was observed that each operator experienced at least some spheroid loss using each of the three addition and removal techniques. However, it was also generally observed that fluid addition had a higher effect on spheroid loss than fluid removal. Further, it was observed that spheroid loss from midpoint notch 108 was greater than for the corner notches 108.

In a third experiment, spheroid retention was again evaluated. Thirteen plates that were ULA-coated were pre-wet using 10 mL of 0.2 μm filtered 35% ethanol, 15 ml of water, and 2×15 mL of DPBS. HT 29/GFP cells were strained through 70 μm cell strainer and seeded at 12×10⁶ cells per plate (about 1,000 cells per cavity of the substrate). Plates were incubated at 37 °C and 5% CO₂ in a humidified incubator. On day three of incubation, one plate was imaged using VMR to provide a control image. The twelve other plates underwent media exchange and were returned to incubation. Four media exchange methods were evaluated, and each method was performed by three operators. The four methods involved: (1) removal and addition of fluid media at a corner notch 108 using a pipette angled transverse to the plane of the substrate 102, (2) removal and addition of fluid media at a corner notch 108 using a pipette angled perpendicular to the plane of the substrate 102, (3) removal of fluid media from a corner notch 108 and addition of fluid media at a midpoint notch 108, using a pipette angled transverse to the plane of the substrate 102, and (4) removal of fluid media from a corner notch 108 and addition of fluid media at a midpoint notch 108, using a pipette angled perpendicular to the plane of the substrate 102. Using these methods by each of the three operators, fluid was exchanged three more times over the next eight days. After the fourth media exchange on the tenth day, the twelve plates were imaged using VMR and analyzed using Halcon to quantify spheroid loss.

From this experiment, it was determined that most of the spheroid loss was in cavities within the first 30% of the area adjacent to the sidewall where the liquid was added. In particular, according to the first method of removing and adding fluid media from the corner notch 108 with an angled pipette, 5% of spheroids (on average between the three microplates) were lost in total, and 73% of the spheroids lost (on average) were located within the first 30% of the substrate 102. According to the second method of removing and adding fluid media from the corner notch 108 using a perpendicular pipette, 4% of spheroids were lost in total, and 46% were located within the first 30% of the substrate 102. According to the third method of removing fluid media from the corner notch 108 and adding fluid media at the midpoint notch 108 using an angled pipette, 6% of spheroids were lost in total, and 68% of the spheroids lost were located within the first 30% of the substrate 102. The results according to the fourth method were the same as the third method. The inventors observed that this experiment confirmed the previous observations that fluid addition has a greater effect on spheroid loss than fluid removal and that spheroid loss was greater when adding fluid media at the midpoint notch 108. It was further determined that the angle of the pipette had little effect at the midpoint notch 108 but that there was about a 1% decrease in spheroid loss when the pipette was at a perpendicular angle for removal/addition at the corner notch 108. Notwithstanding, it was observed that greater than 90% of the spheroids were undisturbed by the exchange of fluid media according to any of the methods, which indicates that the notch (wherever placed) is beneficial in improving yield of 3D cell cultures in open well microplates.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that any particular order be inferred. In addition, as used herein the article “a” is intended include one or more than one component or element, and is not intended to be construed as meaning only one.

It will be apparent to those skilled in the art that various modifications and variations can be made without departing from the spirit or scope of the disclosed embodiments. Since modifications combinations, sub-combinations and variations of the disclosed embodiments incorporating the spirit and substance of the embodiments may occur to persons skilled in the art, the disclosed embodiments should be construed to include everything within the scope of the appended claims and their equivalents.

1. A device for three-dimensional cell growth, comprising:
a substrate comprising an ultra-low attachment surface and a plurality of cavities for growing cell cultures; and
a sidewall surrounding the substrate, the sidewall comprising a first short sidewall, a second short sidewall. a first long sidewall, and a second long sidewall. wherein the first short sidewall and the second short sidewall each comprise a first length, and wherein the first long sidewall and the second long sidewall each comprise a second length. the second length being greater than the first length;
wherein at least one notch is formed on the first short sidewall, the at least one notch configured to accommodate a serological pipette for exchange of fluid media.

2. (canceled)

3. The device of claim 1, wherein the at least one notch comprises a first notch formed on the first short sidewall.

4. The device of claim 3, wherein the first notch is located at a midpoint of the first short sidewall.

5. The device of claim 3, wherein the at least one notch comprises a second notch formed at a corner between the first short sidewall and the first long sidewall.

6. The device of claim 1, wherein the at least one notch comprises a first notch formed at a first corner between the first short sidewall and the first long sidewall.

7. The device of claim 6, wherein the at least one notch comprises a second notch formed at a second corner between the first short sidewall and the second long sidewall.

8. The device of claim 1, wherein the at least one notch comprises substantially planar notch walls.

9. The device of claim 1, wherein the at least one notch comprises a radiused notch wall.

10. The device of claim 1, wherein the at least one notch comprises a floor, wherein the floor is angled relative to a plane defined by the substrate.

11. The device of claim 10, wherein the floor is at a different height than the plane defined by the substrate.

12. The device of claim 1, wherein the plurality of cavities is at least 5 cavities per cm² of the substrate.

13. The device of claim 1, wherein the at least one notch is configured to accommodate the tip of a 25 mL serological pipette oriented perpendicularly to the substrate within the at least one notch.

14. (canceled)

15. A device for three-dimensional cell growth, comprising:
a substrate comprising an ultra-low attachment surface and a plurality of cavities for growing cell cultures;
a single circular sidewall surrounding the substrate; and
wherein at least one notch is formed in the at least one sidewall, the at least one notch configured to accommodate a serological pipette for exchange of fluid media.

16. A method, comprising:
inserting a pipette into a first notch formed in at least one sidewall, the first notch comprising a radiused notch wall and a floor.. the at least one sidewall surrounding a substrate of a cell culture device, the substrate comprising an ultra-low attachment surface and wherein the floor of the first notch is angled relative to a plane defined by the substrate. and a plurality of cavities for growing cell cultures; and
adding a fluid into the first notch such that the fluid covers the substrate within the at least one sidewall.

17. The method of claim 16, wherein the at least one sidewall comprises a first short sidewall, a second short sidewall, a first long sidewall, and a second long sidewall, wherein the first short sidewall and the second short sidewall each comprise a first length, and wherein the first long sidewall and the second long sidewall each comprise a second length, the second length being greater than the first length.

18. The method of claim 17, wherein inserting further comprises inserting the pipette into the first notch, the first notch being formed on the first short sidewall.

19. The method of claim 18, further comprising removing the fluid from over the substrate using a pipette inserted into the first notch.

20. (canceled)

21. The method of claim 18, wherein the first notch is located at a midpoint of the first short sidewall.

22. The method of claim 17, wherein the cell culture device further comprises a second notch and wherein one of the first notch or the second notch is formed on the first short sidewall and the other of the first notch or the second notch is formed at a corner between the first short sidewall and the first long sidewall.

23. The method of claim 22, further comprising raising the second short sidewall, the second short sidewall being opposite to the first short sidewall, such that the fluid flows toward at least one of the first notch or the second notch, removing the fluid from over the substrate using a pipette inserted into the second notch.

24. (canceled)

25. The method of claim 17, wherein inserting further comprises inserting the pipette into the first notch, the first notch being formed at a first corner between the first short sidewall and the first long sidewall.

26. (canceled)

27. The method of claim 16, wherein the at least one notch comprises substantially planar notch walls.

28. (canceled)

29. (canceled)

30. The method of claim 16, wherein the floor is at a different height than the plane defined by the substrate.

31. (canceled)

32. (canceled)

33. (canceled)

34. The method of claim 16, wherein the at least one sidewall is a single circular sidewall.
